(19) United States
(12) Patent Application Publication
(10) Pub. No.: US 2016/0256716 A1
(43) Pub. Date: Sep. 8, 2016

(54) NASAL FILTER

(71) Applicant: Lei WANG, Beijing (CN)

(72) Inventor: Wang Lei, Beijing (CN)

(21) Appl. No.: 15/029,570

(22) PCT Filed: Oct. 17, 2013
(86) PCT No.: PCT/CN2013/085403

§ 371 Date: Apr. 14, 2016

(51) Int. Cl. A62B 23/06; A61M 15/08

(52) U.S. Cl. CPC A62B 23/06; A61M 15/08; A61M 2205/75

(57) ABSTRACT

A nasal filter comprising two cap-shaped structures and a connecting strip. Two extremities of the connecting strip respectively are connected symmetrically to either of the cap-shaped structures. The nasal filter also comprises filter components. The filter components respectively are mounted at the bottom parts of the two cap-shaped structures. Several protrusions are provided on the inner walls of the cap-shaped structures. The bottom parts of the cap-shaped structures are constituted by several cap-shaped structure horizontal beams. The bottom parts and the protrusions jointly jam the filter components. The nasal filter is capable of preventing a filter material from being ejected to the exterior of the cap-shaped structures by air exhaled from the nasal cavities, allows for replacement of the filter material, thus improving the adaptability and economy of the nasal filter in different scenarios.

### TECHNICAL FIELD OF THE INVENTION

The present invention relates to a filtering device, and particularly to a nasal filter.

### BACKGROUND OF THE INVENTION

At present, with conventional nasal filters, a filtering device is inserted into a nostril or used to cover the nostril. When a user inhales air, the filtering device filters particles such as pollen, dust, dander, spore, bacteria and the like out from air. In addition, these nasal filters also perform a function such as sustainedly releasing a volatile medicament and serving as a waterproof nasal plug.

However, the conventional nasal filters have defect in fixation of a filtering material. In some nasal filters, as the filtering material is not adhered to a cap-like structure, the filtering material is not immobilized in the cap-like structure so that the filtering material is apt to be ejected out by air pressure upon breathing and fall off; the filtering material of some other nasal filters is fixed in the cap-like structure in a way of adhesion so that the filtering material cannot be adjusted and replaced, a scope of application of the nasal filters is reduced (if the filtering material is replaceable, filtering material specific for different scenarios may be used), and the cost of use is increased. A glue for adhesion might generate toxic substance or peculiar smell, inhale thereof might affect the user's health and comfortability. In addition, current gas masks for efficiently filtering suspended particles or droplets usually employ multi-layered filtering materials to filter various kinds of harmful substance and improve the filtering effect. For example, activated carbon can filter out harmful gas and peculiar smell, nonwovens can filter out particles suspending in air, and nanometer material can sterilize, thus these materials are superimposed together to filter out various kinds of substance and promote the filtering effect. However, as the nasal filter is usually placed in a nasal passage which has a narrow space, it is very difficult to ensure air permeability while ensuring filtering performance. The existing nasal filters generally employ a single filtering material. However, the signal filtering material has a poor filtering performance relative to the superimposed filtering materials. When various kinds of filtering materials are superimposed and integrated in a way of adhesion, the adhered surfaces will reduce the air permeability of the filtering materials. However, the air permeability is of great importance for the use of the nasal filters.

### SUMMARY OF THE INVENTION

An object of the present invention is to solve the above described technical problems present in the existing nasal filters. The present invention provides a nasal filter which may be used to prevent a filtering material from being ejected out of a cap-like structure by air exhaled from nasal cavity; the filtering material may be replaced very conveniently, thereby promoting its economics and applicability in different scenarios; the nasal filter may employ not only a single filtering material but also a filtering material combined of multi-layers of filtering materials to promote a scope of application and filtering effect of the nasal filter; components of the nasal filter block less part of the air passage and may ensure good air permeability; in addition, the nasal filter is made of a soft material, and components thereof are flexibly connected, enabling comfortable wearing.

To achieve the above described object, the present invention provides a nasal filter comprising two cap-like structures and a connecting band both ends of which are respectively and symmetrically connected with one of the cap-like structures, wherein the nasal filter further comprises filtering members which are respectively mounted on bottoms of the two cap-like structures; inner walls of the cap-like structures are provided with several lugs, the bottoms of the cap-like structures are composed of several cap-like structure cross beams, and the bottoms and the lugs jointly clamp the filtering members.

As a further improvement of the above described technical solution, the filtering members comprise a tray tightly embedded in the cap-like structures and a filtering material which is a single filtering material or multi-layers of filtering materials directly superimposed and disposed in the tray.

As a further improvement of the above described technical solution, the tray comprises a tray annular rim and several tray internally-connected cross beams; the tray annular rim is in a barrel-shaped structure, and the tray internally-connected cross beams are located at a bottom end of the tray annular rim.

As a further improvement of the above described technical solution, the number of the tray internally-connected cross beams is 1-4, and each tray internally-connected cross beam has a width and a thickness both less than 1.5 mm.

As a further improvement of the above described technical solution, the tray is in a barrel-shaped box-like structure, an upper end and a lower end thereof are in a cross beam-like, spoke-like or net-like structure.

As a further improvement of the above described technical solution, the box-like structure comprises a barrel-shaped wall.

As a further improvement of the above described technical solution, the cap-like structures, the connecting band and the trays are all made of soft and nontoxic plastics, resin or silica gel.

As a further improvement of the above described technical solution, the several lugs are all disposed at the same height away from the bottoms of the cap-like structures.

As a further improvement of the above described technical solution, the cross sections of the cap-like structures are in a circular shape, an oval shape or a shape suitable for different inner cavities of nasal vestibules.

As a further improvement of the above described technical solution, the cap-like structures are in a circular truncated cone structure whose ends adjacent to their bottoms have narrower cross sections, and whose ends adjacent to their opening portions have wider cross sections.

As a further improvement of the above described technical solution, the connecting band is in a U-shaped, V-shaped or C-shaped strip structure with both ends being integral with the cap-like structures.

As a further improvement of the above described technical solution, the number of the cap-like structure cross beams is 1-4, and each cap-like cross beam has a width and a thickness both less than 1.5 mm.

As a further improvement of the above described technical solution, the number of the lugs is 3-6.

Advantages of the nasal filter according to the present invention are in that the followings: the inner walls of the cap-like structures in the present invention are provided with several lugs to clamp the filtering members; after the cap-like structures are inserted into the nasal cavity, since opening portions of the cap-like structures are wider and elastic, areas of cross sections thereof will be reduced under pressure from a nostril, which causes areas of the cross sections where the lugs lie to be reduced to further clamp the filtering members tightly, so that the filtering members will not be ejected by the gas exhaled from the nasal cavity; the lugs on the inner walls occupy less space of the air passage so that the filtering members can be replaced more conveniently; trays of the filtering members may enable the use of a combined filtering material so as to promote a scope of application of the nasal filter and improve the filtering effect and the gas permeability, so that the nasal filter becomes a portable gas mask concealed in the nose and is more advantageous than the existing nasal filters employing a single filtering material and the nasal filters combing various kinds of filtering materials in a way of adhesion.

Furthermore, as the tray may be detached easily, the filtering material may be adjusted and replaced conveniently, thus a scope of application and economics of the nasal filter may be promoted; both of the trays and the cap-like structures are made of a soft material, they are in flexible contact with each other and may be flexibly deformed with different shapes of nasal passages, thus the comfortability of the nasal filter is promoted; circular truncated cone-shaped cap-like structures ensure easy insertion thereof into the nasal cavity, the structure with a thick upper portion and a thin bottom portion, in cooperation with the soft material, enhances applicability and airtightness of the nasal filter for nostrils of different sizes; the bottoms of the cap-like structures are provided with several cap-like structure cross beams, on the one hand, the several cap-like structure cross beams together with the lugs of the inner walls of the cap-like structures clamp the filtering members and prevent the filtering members from being drawn in the human body, and on the other hand, the thin cap-like structure cross beams increase air permeability to the largest extent; both ends of the connecting band are integral with the cap-like structures, the structure is simple and will not cause the nasal filter to be drawn in a respiratory tract; the cap-like structures, the connecting band and the trays are all made of nontoxic plastics, resin or silica gel to allow for safe use.

### BRIEF DESCRIPTION OF DRAWINGS

FIG. 1 is a front view of a nasal filter according to an embodiment of the present invention.

FIG. 2 is a rear view of a nasal filter according to an embodiment of the present invention.

FIG. 3 is a sectional view taken along line i-i shown in FIG. 2.

Listing of parts denoted by the following reference numbers:

| | | |
|---|---|---|
| 1. cap-like structure | 2. filtering material | 3. connecting band |
| 4. cap-like structure cross | 5. lug | 6. tray annular rim |
| beam | | |

7. tray internally-connected cross beam

### DETAILED DESCRIPTION OF EMBODIMENTS

The structure of the present invention will be described in more detail in combination with figures and specific embodiments.

A nasal filter according to an embodiment of the present invention comprises two cap-like structures 1 and a connecting band 3 both ends of which are respectively and symmetrically connected with one cap-like structure 1, wherein the nasal filter further comprises filtering members which are respectively mounted on bottoms of the two cap-like structures 1; inner walls of the cap-like structures 1 are provided with several lugs 5, the bottoms of the cap-like structures 1 are composed of several cap-like structure cross beams 4, and the bottoms and the lugs 5 jointly clamp the filtering members.

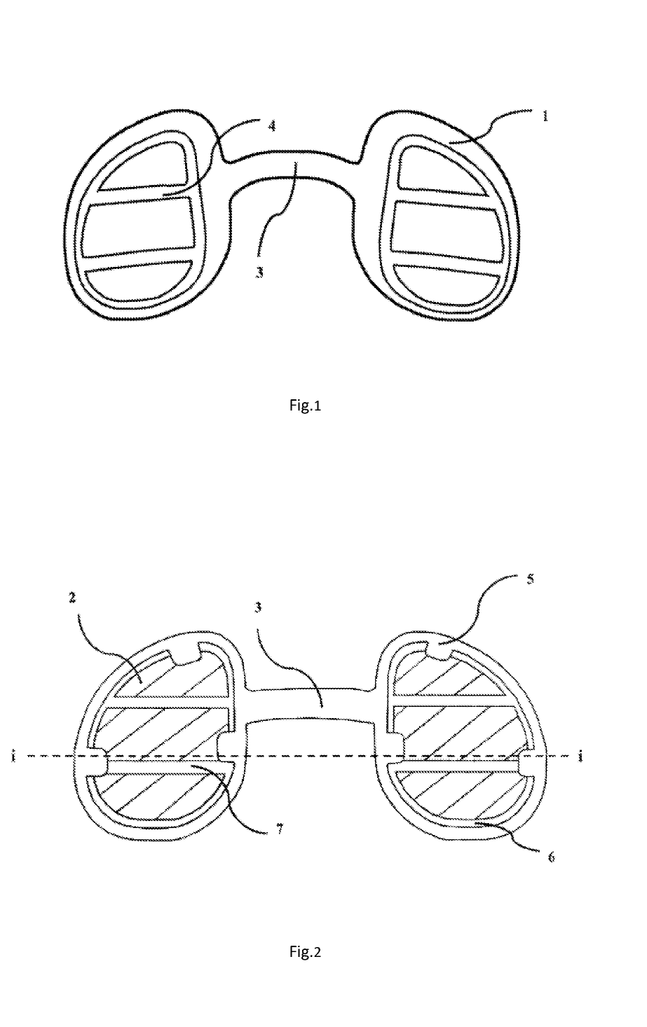

According to the above-described structure of a nasal filter, FIGS. 1 and 2 show a nasal filter according to an embodiment of the present invention. The filtering members comprise a tray and a filtering material 2, the tray is tightly embedded in a cap-like structure 1, the filtering material 2 is a single filtering material or multi-layers of filtering materials directly superimposed and disposed in the tray. In the present embodiment, the tray comprises a tray annular rim 6 and several tray internally-connected cross beams 7; the tray annular rim 6 is in a barrel-shaped structure, and the tray internally-connected cross beams 7 are located at a bottom end of the tray annular rim 6. The number of the tray internally-connected cross beams 7 is 1-4, and each tray internally-connected cross beam 7 has a width and a thickness both less than 1.5 mm. In the present embodiment, the number of the tray internally-connected cross beams 7 is 2. In addition, the tray may be a barrel-shaped box-like structure, an upper end and a lower end thereof are in a cross beam-like, spoke-like or net-like structure; the box-like structure may comprise a barrel-shaped wall.

The several lugs 5 are all disposed at the same height away from the bottom of the cap-like structure 1. A vertical height between the lugs 5 and the cap-like structure cross beams 4 is equal to or slightly smaller than a thickness of the filtering members. A cross section of the cap-like structure 1 may be in a circular shape, an oval shape or a shape suitable for different inner cavities of nasal vestibules. The cap-like structure 1 is in a circular truncated cone structure whose end adjacent to its bottom has a narrower cross section, and whose end adjacent to its opening portion has a wider cross section. The bottom of the cap-like structure 1, when placed in the nasal passage, is oriented towards the bridge of a nose, and the opening portion of the cap-like structure 1, when placed in the nasal passage, is oriented towards the nostril. As shown in FIG. 2, according to the shape of the opening portion of the cap-like structure 1, a side thereof adjacent to a symmetry plane of the nasal filter is a straight edge or an edge with a smaller radian and oriented towards a nasal septum when placed in the nasal passage, a side thereof away from the symmetry plane of the nasal filter is an arcuate edge and oriented towards nose wings when placed in the nasal passage to support an inner wall of the nasal passage. The number of the cap-like structure cross beams 4 is 1-4, each cap-like cross beam 4 has a thickness of less than 1.5 mm, and the number of the lugs 5 is 3-6. Whereas, in the present embodiment, the number of the cross beams 4 is 2; the number of the lugs 5 is 3. In addition, the cap-like structures 1, the connecting band 3 and the trays may be all made of soft and nontoxic plastics, resin or silica gel.

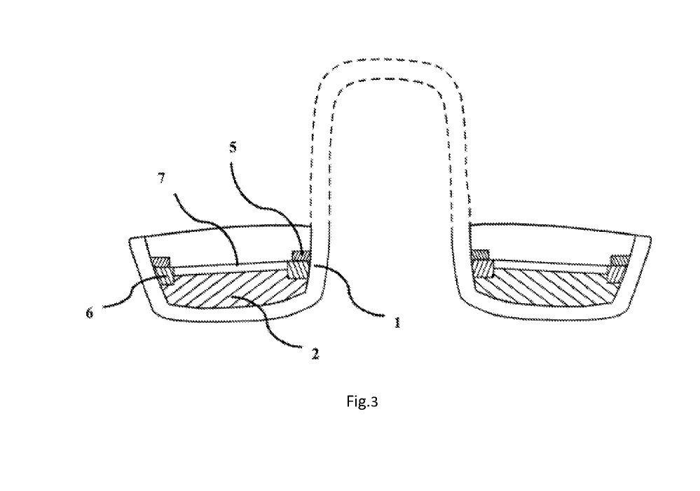

As shown in FIG. 3, the connecting band 3 in the present embodiment is in a U-shaped strip structure, and it may also be in a V-shaped or C-shaped strip structure, both ends of the connecting band 3 are integral with the cap-like structures 1.

Finally it is appreciated that the above embodiments are only intended to illustrate technical solutions of the present invention, not intended for limitation purpose. Although the present invention is described in detail with reference to the embodiments, it should be understood to those persons skilled in the art that modifications or equivalent substitutes to the technical solutions of the present invention all do not depart from the spirit and scope of the technical solutions of the present invention and all shall fall within the scope of claims of the present invention.

1. A nasal filter, comprising two cap-like structures and a connecting band both ends of which are respectively and symmetrically connected with one of the cap-like structures, wherein the nasal filter further comprises filtering members which are respectively mounted on bottoms of the two cap-like structures; inner walls of the cap-like structures are provided with several lugs, the bottoms of the cap-like structures are composed of several cap-like structure cross beams, and the bottoms and the lugs jointly clamp the filtering members.

2. The nasal filter according to claim 1, wherein the filtering members comprise a tray tightly embedded in the cap-like structures and a filtering material which is a single filtering material or multi-layers of filtering materials directly superimposed and disposed in the tray.

3. The nasal filter according to claim 2, wherein the tray comprises a tray annular rim and several tray internally-connected cross beams; the tray annular rim is in a barrel-shaped structure, and the tray internally-connected cross beams are located at a bottom end of the tray annular rim.

4. The nasal filter according to claim 3, wherein the number of the tray internally-connected cross beams is 1-4, and each tray internally-connected cross beam has a width and a thickness both less than 1.5 mm.

5. The nasal filter according to claim 2, wherein the tray is in a barrel-shaped box-like structure, an upper end and a lower end thereof are in a cross beam-like, spoke-like or net-like structure.

6. The nasal filter according to claim 5, wherein the box-like structure comprises a barrel-shaped wall.

7. The nasal filter according to claim 2, wherein the cap-like structures, the connecting band and the trays are all made of soft and nontoxic plastics, resin or silica gel.

8. The nasal filter according to claim 1, wherein several lugs are all disposed at the same height away from the bottoms of the cap-like structures.

9. The nasal filter according to claim 1, wherein the cross sections of the cap-like structures are in a circular shape, an oval shape or a shape suitable for different inner cavities of nasal vestibules.

10. The nasal filter according to claim 1, wherein the cap-like structures are in a circular truncated cone structure whose ends adjacent to their bottoms have narrower cross sections, and whose ends adjacent to their opening portions have wider cross sections.

11. The nasal filter according to claim 1, wherein the connecting band is in a U-shaped, V-shaped or C-shaped strip structure with both ends being integral with the cap-like structures.

12. The nasal filter according to claim 1, wherein the number of the cap-like structure cross beams is 1-4, each cap-like cross beam has a width and a thickness both less than 1.5 mm; and the number of the lugs is 3-6.
